(19) United States
(12) Patent Application Publication
(10) Pub. No.: US 2011/0105492 A1
(43) Pub. Date: May 5, 2011

(54) ARYL SUBSTITUTED ARYLINDENOPYRIMIDINES AND THEIR USE AS HIGHLY SELECTIVE ADENOSINE A2a RECEPTOR ANTAGONISTS

(76) Inventors: Paul F. Jackson, Whitehouse Station, NJ (US); Mark T. Powell, Newtown, PA (US); Brian Christopher Shook, Gilbertsville, PA (US); Aihua Wang, Jamison, PA (US)

(21) Appl. No.: 12/907,299

(22) Filed: Oct. 19, 2010

Related U.S. Application Data
(60) Provisional application No. 61/255,930, filed on Oct. 29, 2009.

(51) Int. Cl. A61K 31/496; C07D 403/10; C07D 239/70; C07D 413/12; A61K 31/517; A61K 31/5377; A61P 25/00; A61P 25/06; A61P 25/16; A61P 25/22; A61P 25/24; A61P 25/28; A61P 25/30

(52) U.S. Cl. 514/232.8; 544/249; 544/115; 514/252.16; 514/267

(57) ABSTRACT

This invention relates to a novel arylindenopyrimidine, A, and its therapeutic and prophylactic uses. Disorders treated and/or prevented include Parkinson's Disease

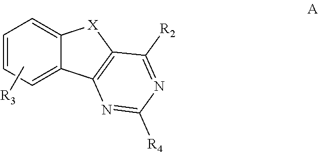

wherein X, R₂, R₃, and R₄ are as defined in the specification.

### CROSS-REFERENCE TO RELATED APPLICATIONS

The present application claims the benefits of the filing of U.S. Provisional Application No. 61/255,930 filed Oct. 29, 2009. The complete disclosures of the aforementioned related patent applications are hereby incorporated herein by reference for all purposes.

### FIELD OF THE INVENTION

This invention relates to aryl substituted arylindenopyrimidines and their therapeutic and prophylactic uses. Disorders treated and/or prevented include neurodegenerative and movement disorders ameliorated by antagonizing Adenosine A_{2A} receptors. The present application is directed to a subset of a genus of compounds, disclosed in U.S. Pat. No. 7,468,373 B2.

### BACKGROUND OF THE INVENTION

Adenosine is a purine nucleotide produced by all metabolically active cells within the body. Adenosine exerts its effects via four subtypes of cell surface receptors (A1, A_{2A}, A2b and A3), which belong to the G protein coupled receptor superfamily. A1 and A3 couple to inhibitory G protein, while A_{2A} and A2b couple to stimulatory G protein. A_{2A} receptors are mainly found in the brain, both in neurons and glial cells (highest level in the striatum and nucleus accumbens, moderate to high level in olfactory tubercle, hypothalamus, and hippocampus etc. regions).

In peripheral tissues, A_{2A} receptors are found in platelets, neutrophils, vascular smooth muscle and endothelium. The striatum is the main brain region for the regulation of motor activity, particularly through its innervation from dopaminergic neurons originating in the substantial nigra. The striatum is the major target of the dopaminergic neuron degeneration in patients with Parkinson's Disease (PD). Within the striatum, A_{2A} receptors are co-localized with dopamine D2 receptors, suggesting an important site for the integration of adenosine and dopamine signaling in the brain.

Adenosine A_{2A} receptor blockers may provide a new class of antiparkinsonian agents (Impagnatiello, F.; Bastia, E.; Ongini, E.; Monopoli, A. Emerging Therapeutic Targets, 2000, 4, 635).

Antagonists of the A_{2A} receptor are potentially useful therapies for the treatment of addiction. Major drugs of abuse (opiates, cocaine, ethanol, and the like) either directly or indirectly modulate dopamine signaling in neurons particularly those found in the nucleus accumbens, which contain high levels of A_{2A} adenosine receptors. Dependence has been shown to be augmented by the adenosine signaling pathway, and it has been shown that administration of an A_{2A} receptor antagonist redues the craving for addictive substances (“The Critical Role of Adenosine A_{2A} Receptors and Gi βγ Subunits in Alcoholism and Addiction: From Cell Biology to Behavior”, by Ivan Diamond and Lina Yao, (The Cell Biology of Addiction, 2006, pp 291-316) and “Adaptations in Adenosine Signaling in Drug Dependence: Therapeutic Implications”, by Stephen P. Hack and Macdonald J. Christie, Critical Review in Neurobiology, Vol. 15, 235-274 (2003)). See also Alcoholism: Clinical and Experimental Research (2007), 31(8), 1302-1307.

An A_{2A} receptor antagonist could be used to treat attention deficit hyperactivity disorder (ADHD) since caffeine (a non selective adenosine antagonist) can be useful for treating ADHD, and there are many interactions between dopamine and adenosine neurons. Clinical Genetics (2000), 58(1), 31-40 and references therein.

A selective A_{2A} antagonist could be used to treat migraine both acutely and prophylactically. Selective adenosine antagonists have shown activity in both acute and prophylactic animal models for migraine (“Effects of K-056, a novel selective adenosine A_{2A} antagonist in animal models of migraine,” by Kurokawa M. et. al., Abstract from Neuroscience 2009).

Antagonists of the A_{2A} receptor are potentially useful therapies for the treatment of depression. A_{2A} antagonists are known to induce activity in various models of depression including the forced swim and tail suspension tests. The positive response is mediated by dopaminergic transmission and is caused by a prolongation of escape-directed behavior rather than by a motor stimulant effect. Neurology (2003), 61(suppl 6) S82-S87.

Antagonists of the A_{2A} receptor are potentially useful therapies for the treatment of anxiety. A_{2A} antagonist have been shown to prevent emotional/anxious responses in vivo. Neurobiology of Disease (2007), 28(2) 197-205.

A_{2A} antagonists have been described in U.S. Pat. No. 7,468,373 B2, US 2009/0054429 A1, and references therein.

### SUMMARY OF THE INVENTION

The genus of compounds disclosed in U.S. Pat. No. 7,468,373 B2 have mixed A_{2A} and A1 receptor antagonism activity. For many disorders for which A_{2A} receptor antagonism is therapeutically useful, the A1 receptor activity is unwanted and may contribute to side effects or even oppose the beneficial effect of the compound primary A_{2A} activity. This invention provides a small group of compounds covered by the genus described in the parent case but that have been found to have surprising and unexpected selectivity for the A_{2A} receptor. The selected group of compounds of the present invention have A_{2A}/A1 activity ratios of at least 50/1, whereas the average member of the genus has an A_{2A}/A1 activity ratio of 1/1. Thus, compounds of the present invention are expected to have much greater therapeutic efficacy and/or fewer side effects.

Selected aryl substituted arylindenopyrimidines of Formula A display unusually high selectivity for A_{2A} over A1 receptor antagonism.

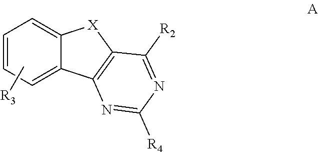

wherein:

### X is C═O;

R₂ is phenyl;

### R₄ is NH₂; and

R₃ is aryl;
said arylindenopyrimidines of Formula A are selected form the group consisting of:

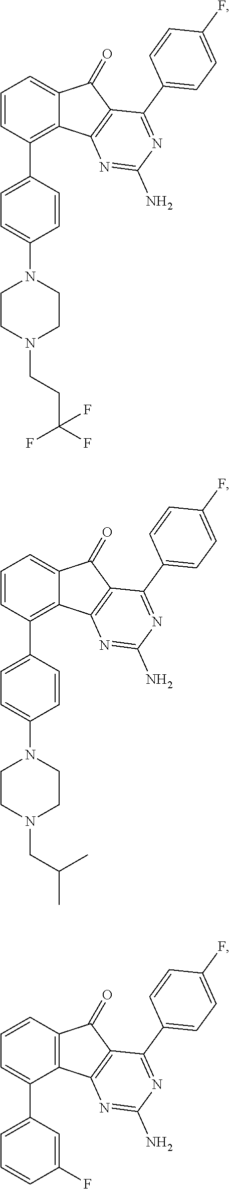

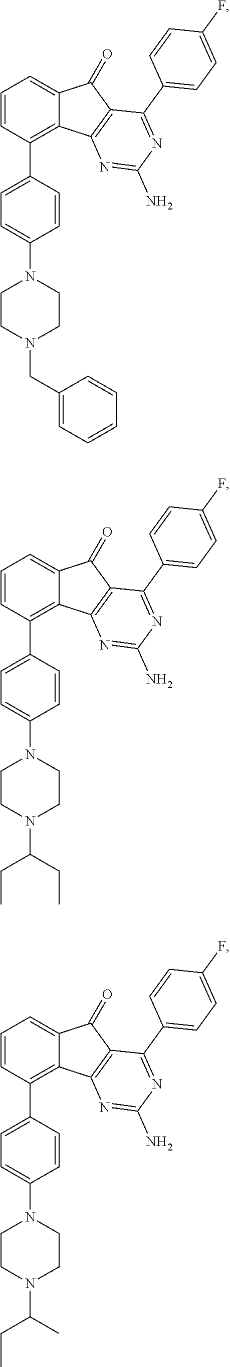

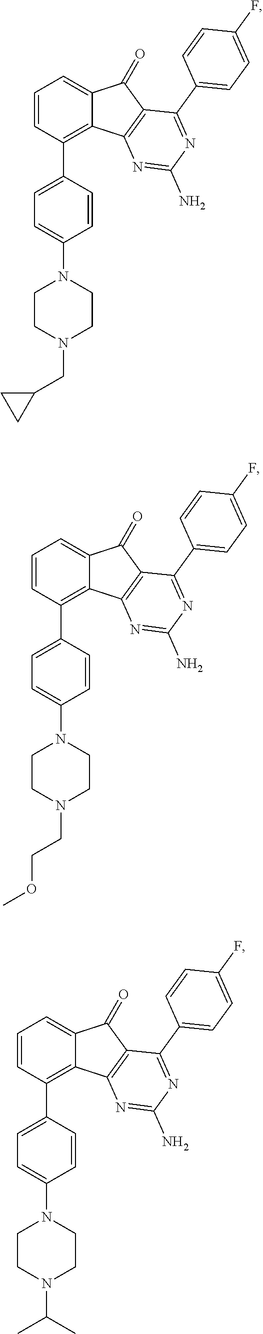

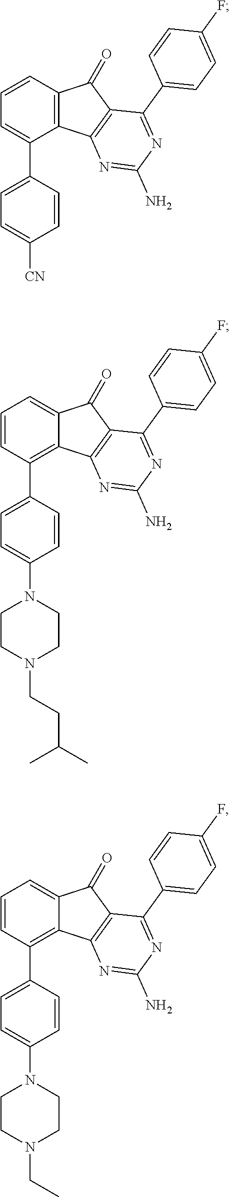

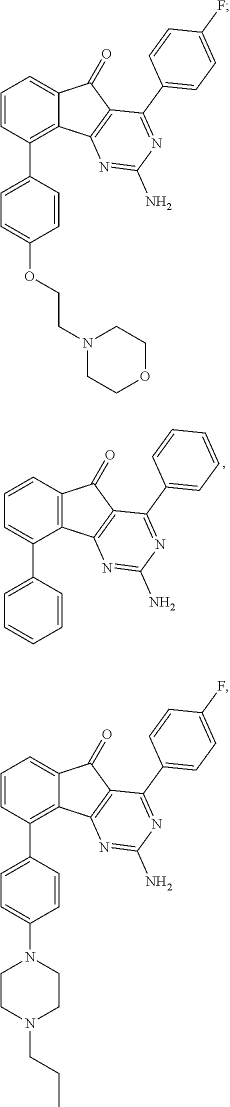

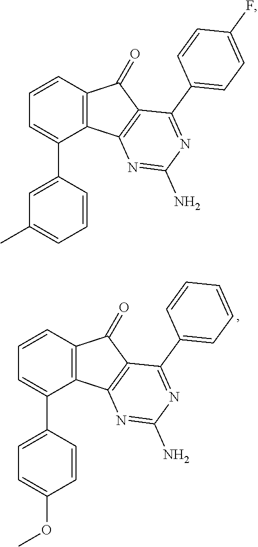

and solvates, hydrates, tautomers, and pharmaceutically acceptable salts thereof;

### DETAILED DESCRIPTION OF THE INVENTION

The invention provides arylindenopyrimidines of Formula A JNJ-39928122.

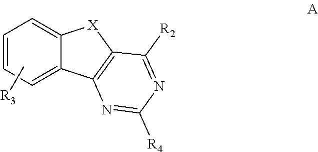

wherein:

### X is C═O;

R₂ is phenyl;

### R₄ is NH₂; and

R₃ is aryl;
said arylindenopyrimidines of Formula A are selected form the group consisting of:

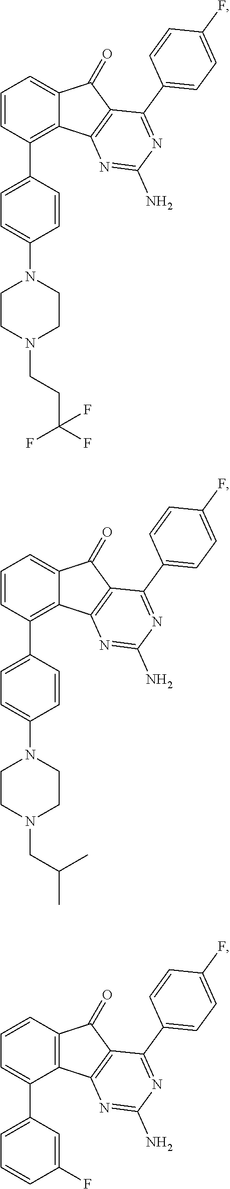

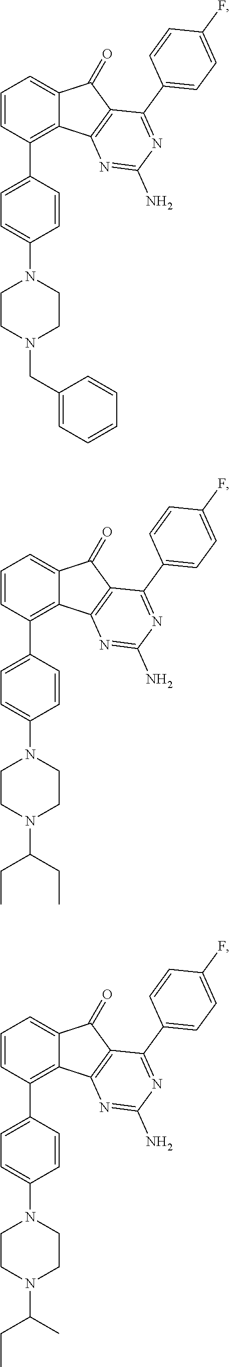

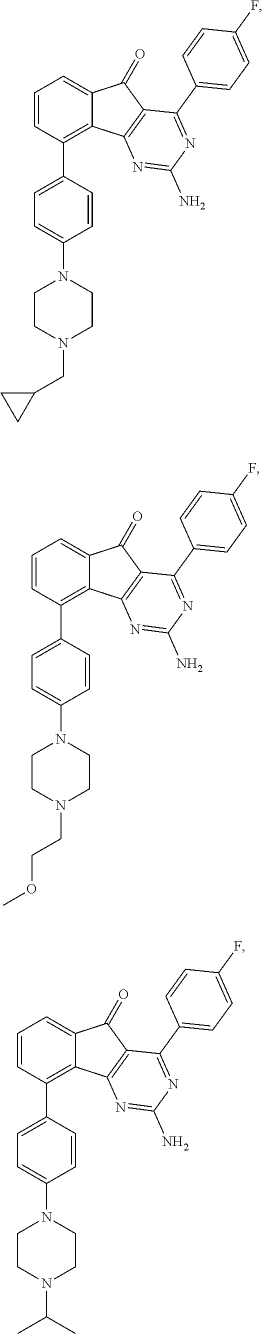

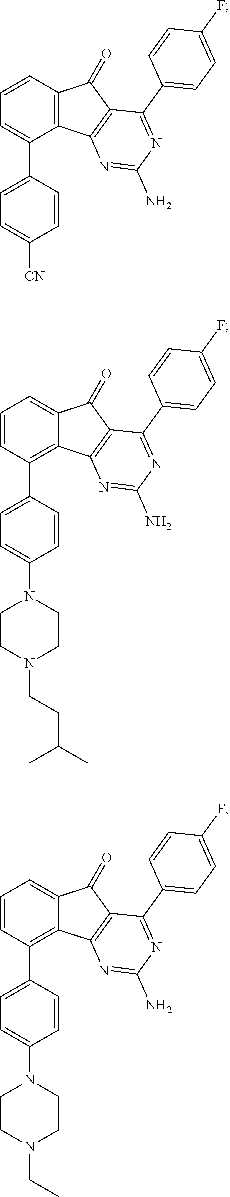

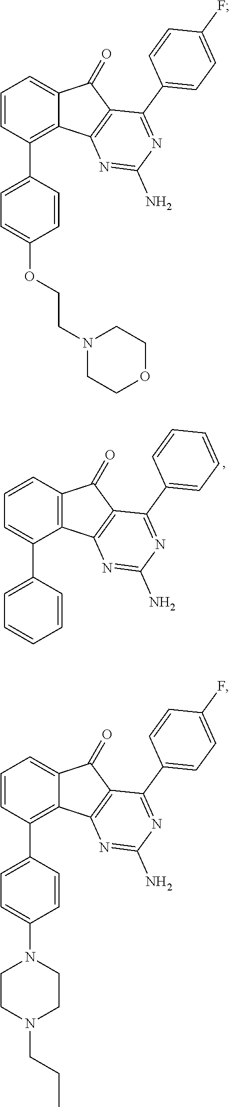

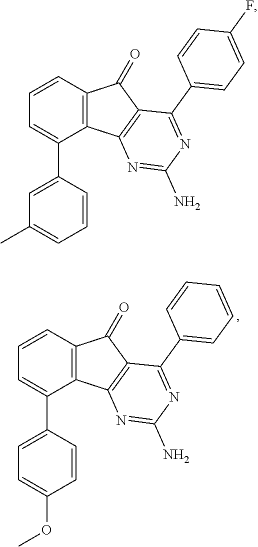

and solvates, hydrates, tautomers, and pharmaceutically acceptable salts thereof;

This invention further provides a method of treating a subject having a disorder ameliorated by antagonizing Adenosine A_{2A} receptors, which comprises administering to the subject a therapeutically effective dose of a compound of claim 1.

This invention further provides a method of preventing a disorder ameliorated by antagonizing Adenosine A_{2A} receptors in a subject, comprising of administering to the subject a prophylactically effective dose of a compound of claim 1 either preceding or subsequent to an event anticipated to cause a disorder ameliorated by antagonizing Adenosine A_{2A} receptors in the subject.

The instant compounds can be isolated and used as free bases. They can also be isolated and used as pharmaceutically acceptable salts.

Examples of such salts include hydrobromic, hydroiodic, hydrochloric, perchloric, sulfuric, maleic, fumaric, malic, tartaric, citric, adipic, benzoic, mandelic, methanesulfonic, hydroethanesulfonic, benzenesulfonic, oxalic, palmoic, 2 naphthalenesulfonic, p-toluenesulfonic, cyclohexanesulfamic and saccharic.

This invention also provides a pharmaceutical composition comprising a compound of claim 1 and a pharmaceutically acceptable carrier.

Pharmaceutically acceptable carriers are well known to those skilled in the art and include, but are not limited to, from about 0.01 to about 0.1 M and preferably 0.05 M phosphate buyer or 0.8% saline. Such pharmaceutically acceptable carriers can be aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers include water, ethanol, alcoholic/aqueous solutions, glycerol, emulsions or suspensions, including saline and buffered media. Oral carriers can be elixirs, syrups, capsules, tablets and the like. The typical solid carrier is an inert substance such as lactose, starch, glucose, methyl-cellulose, magnesium stearate, dicalcium phosphate, mannitol and the like. Parenteral carriers include sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like.

Preservatives and other additives can also be present, such as, for example, antimicrobials, antioxidants, chelating agents, inert gases and the like. All carriers can be mixed as needed with disintegrants, diluents, granulating agents, lubricants, binders and the like using conventional techniques known in the art.

This invention further provides a method of treating a subject having a condition ameliorated by antagonizing Adenosine A_{2A} receptors, which comprises administering to the subject a therapeutically effective dose of a compound of claim 1.

In one embodiment, the disorder is a neurodegenerative or movement disorder. Examples of disorders treatable by the instant pharmaceutical composition include, without limitation, Parkinson's Disease, Huntington's Disease, Multiple System Atrophy, Corticobasal Degeneration, Alzheimer's Disease, and Senile Dementia.

In one preferred embodiment, the disorder is Parkinson's disease.

As used herein, the term “subject” includes, without limitation, any animal or artificially modified animal having a disorder ameliorated by antagonizing adenosine A_{2A} receptors. In a preferred embodiment, the subject is a human.

Administering a compound of claim 1 can be effected or performed using any of the various methods known to those skilled in the art. The compounds of claim 1 can be administered, for example, intravenously, intramuscularly, orally and subcutaneously.

In the preferred embodiment, compounds of claim 1 are administered orally. Additionally, administration can comprise giving the subject a plurality of dosages over a suitable period of time. Such administration regimens can be determined according to routine methods.

As used herein, a “therapeutically effective dose” of a pharmaceutical composition is an amount sufficient to stop, reverse or reduce the progression of a disorder. A “prophylactically effective dose” of a pharmaceutical composition is an amount sufficient to prevent a disorder, i.e., eliminate, ameliorate and/or delay the disorder's onset. Methods are known in the art for determining therapeutically and prophylactically effective doses for compounds of claim 1. The effective dose for administering the pharmaceutical composition to a human, for example, can be determined mathematically from the results of animal studies.

In one embodiment, the therapeutically and/or prophylactically effective dose is a dose sufficient to deliver from about 0.001 mg/kg of body weight to about 200 mg/kg of body weight of a compound of claim 1. In another embodiment, the therapeutically and/or prophylactically effective dose is a dose sufficient to deliver from about 0.05 mg/kg of body weight to about 50 mg/kg of body weight. More specifically, in one embodiment, oral doses range from about 0.05 mg/kg to about 100 mg/kg daily. In another embodiment, oral doses range from about 0.05 mg/kg to about 50 mg/kg daily, and in a further embodiment, from about 0.05 mg/kg to about 20 mg/kg daily. In yet another embodiment, infusion doses range from about 1.0 μg/kg/min to about 10 mg/kg/min of inhibitor, admixed with a pharmaceutical carrier over a period ranging from about several minutes to about several days. In a further embodiment, for topical administration, the instant compound can be combined with a pharmaceutical carrier at a drug/carrier ratio of from about 0.001 to about 0.1.

The invention also provides a method of treating addiction in a mammal, comprising administering a therapeutically effective dose of a compound of claim 1.

The invention also provides a method of treating ADHD in a mammal, comprising administering a therapeutically effective dose of a compound of claim 1.

The invention also provides a method of treating depression in a mammal, comprising administering a therapeutically effective dose of a compound of claim 1.

The invention also provides a method of treating anxiety in a mammal, comprising administering a therapeutically effective dose of a compound of claim 1.

The invention also provides a method of treating migraine in a mammal, comprising administering a therapeutically effective dose of a compound of claim 1.

### DEFINITIONS AND NOMENCLATURE

Unless otherwise noted, under standard nomenclature used throughout this disclosure the terminal portion of the designated side chain is described first, followed by the adjacent functionality toward the point of attachment.

As used herein, the following chemical terms shall have the meanings as set forth in the following paragraphs: “independently”, when in reference to chemical substituents, shall mean that when more than one substituent exists, the substituents may be the same or different.

“Alkyl” shall mean straight, cyclic and branched-chain alkyl. Unless otherwise stated, the alkyl group will contain 1-20 carbon atoms. Unless otherwise stated, the alkyl group may be optionally substituted with one or more groups such as halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, carboxamide, hydroxamic acid, sulfonamide, sulfonyl, thiol, aryl, aryl(c₁-c₈)alkyl, heterocyclyl, and heteroaryl.

“Alkoxy” shall mean —O-alkyl and unless otherwise stated, it will have 1-8 carbon atoms.

“Halogen” shall mean fluorine, chlorine, bromine or iodine; “PH” or “Ph” shall mean phenyl; “Ac” shall mean acyl; “Bn” shall mean benzyl.

The term “acyl” as used herein, whether used alone or as part of a substituent group, means an organic radical having 2 to 6 carbon atoms (branched or straight chain) derived from an organic acid by removal of the hydroxyl group. The term “Ac” as used herein, whether used alone or as part of a substituent group, means acetyl.

“Aryl” or “Ar,” whether used alone or as part of a substituent group, is a carbocyclic aromatic radical including, but not limited to, phenyl, 1- or 2-naphthyl and the like. The carbocyclic aromatic radical may be substituted by independent replacement of 1 to 5 of the hydrogen atoms thereon with halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, or carboxamide. Illustrative aryl radicals include, for example, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl and the like. “Ph” or “PH” denotes phenyl.

Whether used alone or as part of a substituent group, “heteroaryl” refers to a cyclic, fully unsaturated radical having from five to ten ring atoms of which one ring atom is selected from S, O, and N; 0-2 ring atoms are additional heteroatoms independently selected from S, O, and N; and the remaining ring atoms are carbon. The radical may be joined to the rest of the molecule via any of the ring atoms. Exemplary heteroaryl groups include, for example, pyridinyl, pyrazinyl, pyrimidinyl, pyridazinyl, pyrroyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, 2-oxazepinyl, azepinyl, N-oxo-pyridyl, 1-dioxothienyl, benzothiazolyl, benzoxazolyl, benzothienyl, quinolinyl-N-oxide, benzimidazolyl, benzopyranyl, benzisothiazolyl, benzisoxazolyl, benzodiazinyl, benzofurazanyl, benzothiopyranyl, indazolyl, indolizinyl, benzofuryl, chromonyl, coumarinyl, cinnolinyl, quinoxalinyl, indazolyl, pyrrolopyridinyl, furopyridinyl (such as furo[2,3-c]pyridinyl, furo[3,2-b]pyridinyl, or furo[2,3-b]pyridinyl), imidazopyridinyl (such as imidazo[4,5-b]pyridinyl or imidazo[4,5-c]pyridinyl), naphthyridinyl, phthalazinyl, purinyl, pyridopyridyl, quinazolinyl, thienofuryl, thienopyridyl, thienothienyl, and furyl. The heteroaryl group may be substituted by independent replacement of 1 to 5 of the hydrogen atoms thereon with halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, or carboxamide. Heteroaryl may be substituted with a mono-oxo to give for example a 4-oxo-1H-quinoline.

The terms “heterocycle,” “heterocyclic,” and “heterocyclo” refer to an optionally substituted, fully or partially saturated cyclic group which is, for example, a 4- to 7-membered monocyclic, 7- to 1′-membered bicyclic, or 10- to 15-membered tricyclic ring system, which has at least one heteroatom in at least one carbon atom containing ring. Each ring of the heterocyclic group containing a heteroatom may have 1, 2, or 3 heteroatoms selected from nitrogen atoms, oxygen atoms, and sulfur atoms, where the nitrogen and sulfur heteroatoms may also optionally be oxidized. The nitrogen atoms may optionally be quaternized. The heterocyclic group may be attached at any heteroatom or carbon atom.

Exemplary monocyclic heterocyclic groups include pyrrolidinyl; oxetanyl; pyrazolinyl; imidazolinyl; imidazolidinyl; oxazolyl; oxazolidinyl; isoxazolinyl; thiazolidinyl; isothiazolidinyl; tetrahydrofuryl; piperidinyl; piperazinyl; 2-oxopiperazinyl; 2-oxopiperidinyl; 2-oxopyrrolidinyl; 4-piperidonyl; tetrahydropyranyl; tetrahydrothiopyranyl; tetrahydrothiopyranyl sulfone; morpholinyl; thiomorpholinyl; thiomorpholinyl sulfoxide; thiomorpholinyl sulfone; 1,3-dioxolane; dioxanyl; thietanyl; thiiranyl; and the like. Exemplary bicyclic heterocyclic groups include quinuclidinyl; tetrahydroisoquinolinyl; dihydroisoindolyl; dihydroquinazolinyl (such as 3,4-dihydro-4-oxo-quinazolinyl); dihydrobenzofuryl; dihydrobenzothienyl; dihydrobenzothiopyranyl; dihydrobenzothiopyranyl sulfone; dihydrobenzopyranyl; indolinyl; isochromanyl; isoindolinyl; piperonyl; tetrahydroquinolinyl; and the like.

Substituted aryl, substituted heteroaryl, and substituted heterocycle may also be substituted with a second substituted-aryl, a second substituted-heteroaryl, or a second substituted-heterocycle to give, for example, a 4-pyrazol-1-yl-phenyl or 4-pyridin-2-yl-phenyl.

Designated numbers of carbon atoms (e.g., C₁₋₈) shall refer independently to the number of carbon atoms in an alkyl or cycloalkyl moiety or to the alkyl portion of a larger substituent in which alkyl appears as its prefix root.

### EXAMPLES

Compounds of Formula A can be prepared by methods known to those who are skilled in the art. The following reaction scheme is only meant to represent an example of the invention and is in no way meant to limit the invention.

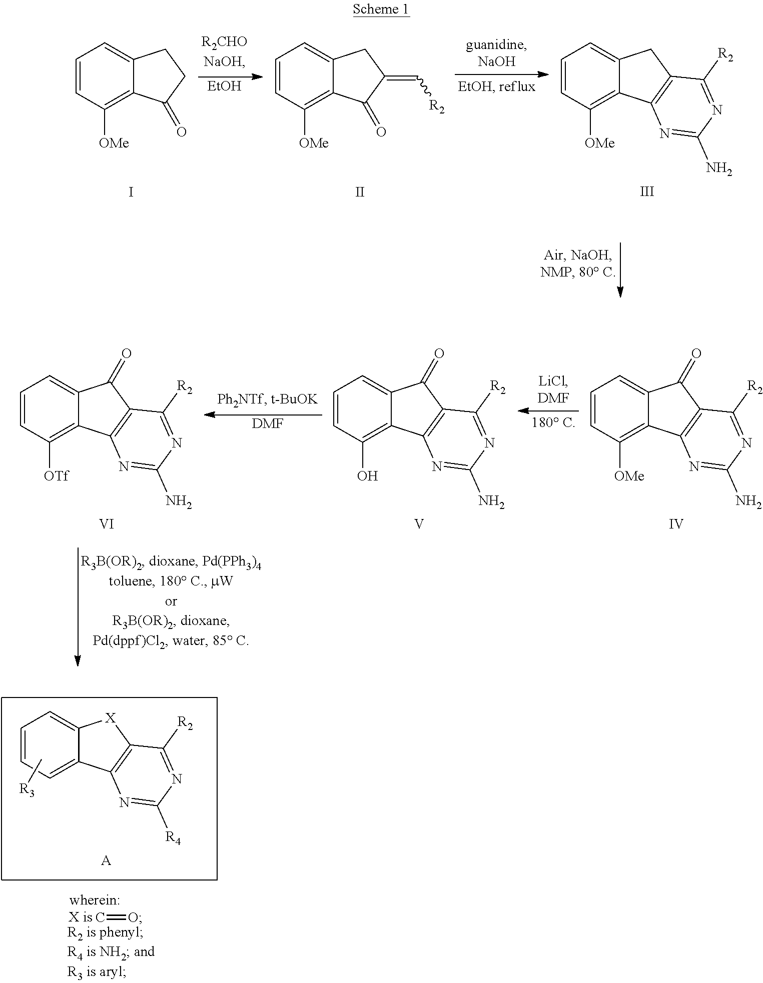

Scheme 1 illustrates the synthetic route leading to compound A. Starting with 7-methoxy indanone I and following the path indicated by the arrows, condensation under basic conditions with arylaldehydes affords the benzylidene II. The benzylidene II is then reacted with guanidine (free base) that gives the intermediate amino pyrimidine III and is directly oxidized to the corresponding ketone IV by bubbling air through the basic N-methylpyrrolidinone (NMP) solution. Demethylation can be accomplished by heating IV in NMP in the presence of LiCl to give the corresponding phenol V. The phenol V can be converted to corresponding triflate VI by treatment with N-phenyltriflimide under basic conditions in dimethylformamide (DMF). Finally, the triflate VI is reacted with boronic esters of formula R²B(OR)₂ to afford compounds of formula A.

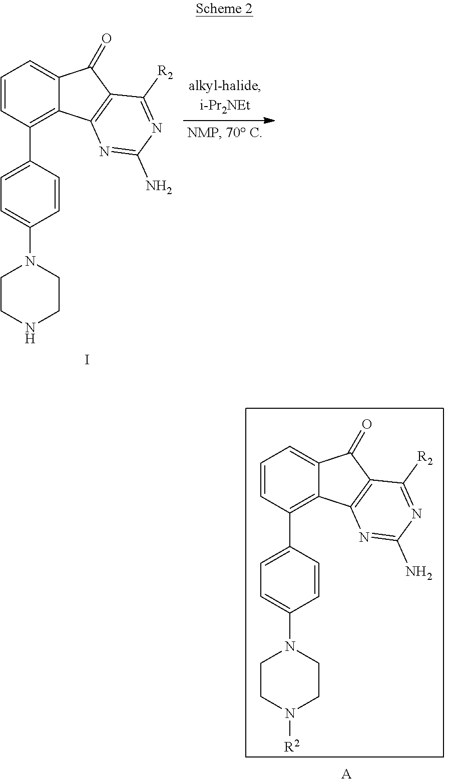

Scheme 2 illustrates the synthetic route leading to compounds of formula A, where R₃ is an alkylpiperazinyl substituted phenyl. Starting from piperazine I, prepared according to scheme 1, is alkylated with alkyl halides in N-methylpyrrolidinone (NMP) to afford compounds of formula A.

### Example 1

### 2-Amino-4-(4-fluoro-phenyl)-9-{4-[4-(3,3,3-trifluoro-propyl)-piperazin-1-yl]-phenyl}-indeno[1,2-d]pyrimidin-5-one

### Example 1

### Step a

### 2-(4-Fluoro-benzylidene)-7-methoxy-indan-1-one

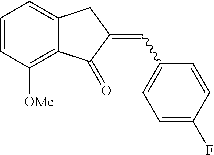

An aqueous solution (2 mL) of NaOH (615 mg, 15.4 mmol) was added dropwise to an ethanol (EtOH) solution (13 mL) of 7-methoxy-indan-1-one (2.0 g, 12.3 mmol) and 4-fluoro-benzaldehyde (1.4 mL, 12.9 mmol). A precipitate formed immediately. The resulting slurry was stirred vigorously for 0.5 h. The slurry was cooled in an ice bath, filtered, and washed with cold EtOH. The collected solid was dried in vacuo to give the title compound that was used without further purification.

### Example 1

### Step b

### 4-(4-Fluoro-phenyl)-9-methoxy-5H-indeno[1,2-d]pyrimidin-2-ylamine

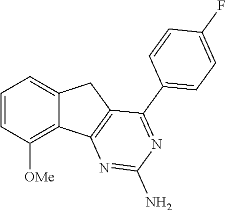

Powdered NaOH (2.5 g, 62.5 mmol) was added to an EtOH solution (50 mL) of guanidine hydrochloride (5.9 g, 61.6 mmol). After 30 min the sodium chloride was filtered off and the filtrate was added to an EtOH suspension (20 mL) of 2-(4-fluoro-benzylidene)-7-methoxy-indan-1-one (3.3 g, 12.3 mmol). The resulting mixture was heated to reflux overnight. The homogeneous solution was cooled in ice for 30 minutes and filtered to give the title compound which was used without further purification.

### Example 1

### Step c

### 2-Amino-4-(4-fluoro-phenyl)-9-methoxy-indeno[1,2-d]pyrimidin-5-one

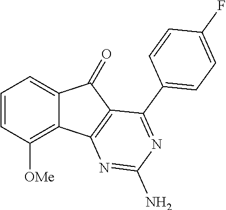

Powdered NaOH (96 mg, 2.4 mmol) was added to a NMP solution (10 mL) of 4-(4-Fluoro-phenyl)-9-methoxy-5H-indeno[1,2-d]pyrimidin-2-ylamine (740 mg, 2.4 mmol). The resulting mixture was heated to 80° C. and air was bubbled through the solution. After 16 hours the mixture was cooled to room temperature, water was added and the resulting precipitate was filtered and washed with water and cold EtOH. The solid was dried in vacuo to give the title compound that was used without further purification.

### Example 1

### Step d

### 2-Amino-4-(4-fluoro-phenyl)-9-hydroxy-indeno[1,2-d]pyrimidin-5-one

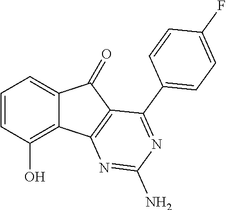

Solid LiCl (384 mg, 9.1 mmol) was added to an NMP solution (2.5 mL) of 2-amino-4-(4-fluoro-phenyl)-9-methoxy-indeno[1,2-d]pyrimidin-5-one (485 mg, 1.5 mmol) and water (0.05 mL) and the mixture was heated to 180° C. in the microwave. After 2 hours the mixture was diluted with THF and EtOAc, washed with water and brine, dried (Na₂SO₄), and dry packed onto silica gel. Chromatography gave the title compound.

### Example 1

### Step e

### Trifluoro-methanesulfonic acid 2-amino-4-(4-fluoro-phenyl)-5-oxo-5H-indeno[1,2-d]pyrimidin-9-yl ester

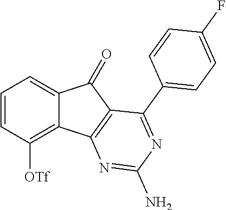

Solid t-BuOK (potassium tert-butoxide, 877 mg, 7.8 mmol) was added to a DMF solution (30 mL) of 2-amino-4-(4-fluoro-phenyl)-9-hydroxy-indeno[1,2-d]pyrimidin-5-one (2.0 g, 6.5 mmol). After 20 min, solid PhN(Tf)₂ (phenyl bis(trifluoromethane)sulfonamide, 2.5 g, 6.8 mmol) was added. After 3 hours water was added and the resulting precipitate was filtered off and washed with water. The solid was dissolved in THF and dry packed onto silica gel. Column chromatography gave the title compound.

### Example 1

### Step f

### 2-Amino-4-(4-fluoro-phenyl)-9-(4-piperazin-1-yl-phenyl)-indeno[1,2-d]pyrimidin-5-one

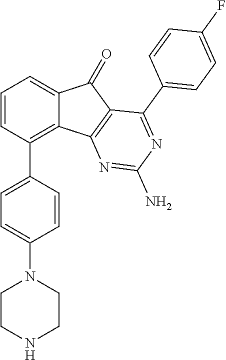

Solid Pd(dppf)Cl₂ (dichloro[1,1′-ferrocenylbis(diphenyl-phosphine)]palladium(II), 47 mg, 0.06 mmol) was added to a dioxane/water solution (4 mL/1 mL) of 1-[4-(4,4,5,5-tetramethyl-[1,3,2]dioxaborolan-2-yl)-phenyl]-piperazine (213 mg, 0.75 mmol), trifluoro-methanesulfonic acid 2-amino-4-(4-fluoro-phenyl)-5-oxo-5H-indeno[1,2-d]pyrimidin-9-yl ester (250 mg, 0.57 mmol), and K₂CO₃(158 mg, 1.14 mmol) and the mixture was heated to 85° C. After 5 hours the mixture was cooled, diluted with water and the resulting precipitate was filtered. The collected solid was dissolved in THF and MeOH then dry packed onto silica gel. Column chromatography gave the title compound.

### Example 1

### Step g

### 2-Amino-4-(4-fluoro-phenyl)-9-{4-[4-(3,3,3-trifluoro-propyl)-piperazin-1-yl]-phenyl}-indeno[1,2-d]pyrimidin-5-one

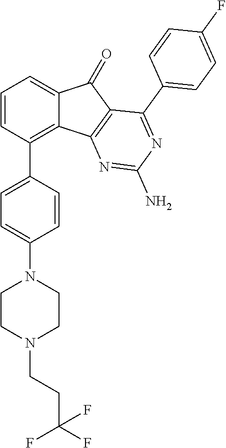

Neat 1,1,1-trifluoro-3-iodo-propane was added to an NMP solution (10 mL) of 2-amino-4-(4-fluoro-phenyl)-9-(4-piperazin-1-yl-phenyl)-indeno[1,2-d]pyrimidin-5-one (1.4 g, 2.7 mmol) and i-Pr₂NEt (2.3 mL, 13.3 mmol) and the mixture was heated to 70° C. After 16 hours the mixture was cooled, diluted with water and the resulting precipitate was filtered. The collected solid was dissolved in THF and dry packed onto silica gel. Column chromatography gave the title compound. ¹H NMR (CHLOROFORM-d, 300 MHz): δ=8.04-8.13 (m, 2H), 7.70 (dd, J=6.8, 1.5 Hz, 1 H), 7.45-7.59 (m, 4H), 7.12-7.22 (m, 2H), 7.00 (d, J=8.7 Hz, 2H), 5.47 (br. s., 2 H), 3.27-3.37 (m, 4H), 2.62-2.75 (m, 6H), 2.28-2.48 ppm (m, 2H); MS m/e 548 (M+H).

### Example 2

### 2-Amino-4-(4-fluoro-phenyl)-9-[4-(4-isobutyl-piperazin-1-yl)-phenyl]-indeno[1,2-d]pyrimidin-5-one

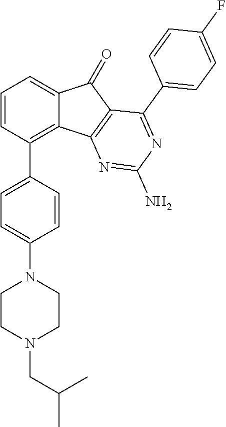

The title compound was prepared using 1-iodo-2-methyl-propane in place of 1,1,1-trifluoro-3-iodo-propane as described in Example 1. ¹H NMR(CHLOROFORM-d, 300 MHz): δ=8.01-8.16 (m, 2H), 7.69 (dd, J=6.6, 1.7 Hz, 1H), 7.46-7.60 (m, 4 H), 7.10-7.23 (m, 2H), 7.00 (d, J=9.0 Hz, 2H), 5.48 (br. s., 2H), 3.22-3.41 (m, 4 H), 2.52-2.68 (m, 4H), 2.16 (d, J=7.5 Hz, 2H), 1.84 (dt, J=13.6, 6.8 Hz, 1H), 0.94 ppm (d, J=6.4 Hz, 6H); MS m/e 508 (M+H).

### Example 3

### 2-Amino-4-(4-fluoro-phenyl)-9-(3-fluoro-phenyl)-indeno[1,2-d]pyrimidin-5-one

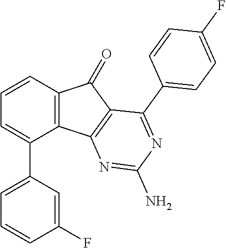

A solution of trifluoro-methanesulfonic acid 2-amino-4-(4-fluoro-phenyl)-5-oxo-5H-indeno[1,2-d]pyrimidin-9-yl ester (prepared as described in Example 1) (150 mg, 0.34 mmol), 3-fluoro-phenylboronic acid (70 mg, 0.51 mmol), (PPh₃)₄Pd (tetrakis(triphenylphosphine)palladium(0), 20 mg, 0.02 mmol), and K₂CO₃ (99 mg, 0.72 mmol) in dioxane (1 mL) and toluene (1 mL) was heated to 180° C. by microwave irradiation. After 30 min the mixture was cooled to room temperature, and purified via column chromatography to give the title compound. ¹H NMR (DMSO-d₆, 400 MHz): δ=8.00-8.07 (m, 2H), 7.64-7.73 (m, 2H), 7.58 (dd, J=5.4, 3.4 Hz, 1H), 7.40-7.53 (m, 3H), 7.29-7.37 (m, 2H), 7.26 ppm (d, J=1.2 Hz, 1H); MS m/e 386 (M+H).

### Example 4

### 2-Amino-9-[4-(4-benzyl-piperazin-1-yl)-phenyl]-4-(4-fluoro-phenyl)-indeno[1,2-d]pyrimidin-5-one

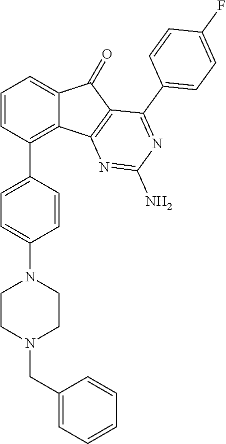

The title compound was prepared using bromomethyl-benzene in place of 1,1,1-trifluoro-3-iodo-propane as described in Example 1. ¹H NMR(CHLOROFORM-d, 300 MHz): δ=8.04-8.13 (m, 2H), 7.69 (dd, J=6.6, 1.7 Hz, 1H), 7.46-7.58 (m, 4 H), 7.33-7.41 (m, 4H), 7.17 (t, J=8.9 Hz, 3H), 6.99 (d, J=8.7 Hz, 2H), 5.42 (br. s., 2H), 3.61 (s, 2H), 3.33 (t, J=4.9 Hz, 4H), 2.58-2.72 ppm (m, 4H); MS m/e 542 (M+H).

### Example 5

### 2-Amino-9-{4-[4-(1-ethyl-propyl)-piperazin-1-yl]-phenyl}-4-(4-fluoro-phenyl)-indeno[1,2-d]pyrimidin-5-one

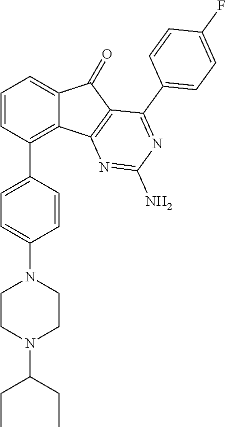

The title compound was prepared using 3-bromo-pentane in place of 1,1,1-trifluoro-3-iodo-propane as described in Example 1. ¹H NMR(CHLOROFORM-d, 300 MHz): δ=8.05-8.14 (m, 2H), 7.69 (dd, J=6.4, 1.9 Hz, 1H), 7.47-7.57 (m, 4H), 7.12-7.22 (m, 2H), 7.00 (d, J=9.0 Hz, 2H), 5.45 (br. s., 2H), 3.22-3.35 (m, 4H), 2.72 (br. s., 4H), 2.24 (s, 1H), 1.38 (d, J=7.5 Hz, 2H), 1.20-1.30 (m, 2H), 0.94 ppm (t, J=7.3 Hz, 6H); MS m/e 522 (M+H).

### Example 6

### 2-Amino-9-[4-(4-sec-butyl-piperazin-1-yl)-phenyl]-4-(4-fluoro-phenyl)-indeno[1,2-d]pyrimidin-5-one

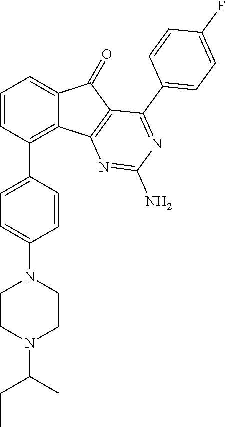

The title compound was prepared using 2-bromo-butane in place of 1,1,1-trifluoro-3-iodo-propane as described in Example 1. ¹H NMR(CHLOROFORM-d, 300 MHz): δ=8.01-8.14 (m, 2H), 7.69 (dd, J=6.4, 1.9 Hz, 1H), 7.45-7.60 (m, 4H), 7.11-7.22 (m, 2H), 7.00 (d, J=9.0 Hz, 2H), 5.47 (br. s., 2H), 3.30 (t, J=4.9 Hz, 4H), 2.62-2.82 (m, 4H), 2.52 (br. s., 1H), 1.61-1.71 (m, 1H), 1.28-1.43 (m, 1H), 1.04 (d, J=6.8 Hz, 3H), 0.94 ppm (t, J=7.5 Hz, 3H); MS m/e 508 (M+H).

### Example 7

### 2-Amino-9-[4-(4-cyclopropylmethyl-piperazin-1-yl)-phenyl]-4-(4-fluoro-phenyl)-indeno[1,2-d]pyrimidin-5-one

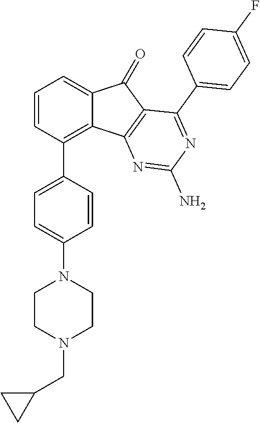

The title compound was prepared using bromomethyl-cyclopropane in place of 1,1,1-trifluoro-3-iodo-propane as described in Example 1. ¹H NMR(CHLOROFORM-d, 300 MHz): δ=8.02-8.14 (m, 2H), 7.70 (dd, J=6.8, 1.9 Hz, 1H), 7.47-7.59 (m, 4H), 7.12-7.22 (m, 2H), 7.01 (d, J=8.7 Hz, 2H), 5.44 (br. s., 2H), 3.31-3.43 (m, 4H), 2.68-2.86 (m, 4H), 2.38 (br. s., 2H), 0.88-1.02 (m, 1H), 0.51-0.65 (m, 2H), 0.13-0.21 ppm (m, 2H); MS m/e 506 (M+H).

### Example 8

### 2-Amino-4-(4-fluoro-phenyl)-9-{4-[4-(2-methoxy-ethyl)-piperazin-1-yl]-phenyl}-indeno[1,2-d]pyrimidin-5-one

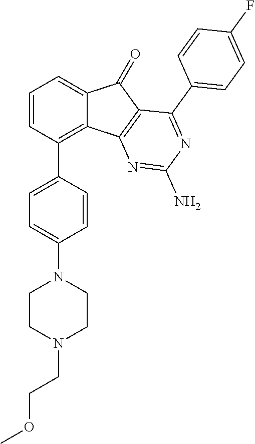

The title compound was prepared using 1-bromo-2-methoxy-ethane in place of 1,1,1-trifluoro-3-iodo-propane as described in Example 1. ¹H NMR(CHLOROFORM-d, 300 MHz): δ=8.04-8.15 (m, 2H), 7.70 (dd, J=6.8, 1.9 Hz, 1H), 7.47-7.59 (m, 4H), 7.13-7.22 (m, 2H), 7.00 (d, J=9.0 Hz, 2H), 5.47 (br. s., 2H), 3.61 (t, J=5.5 Hz, 2H), 3.39 (s, 3H), 3.32-3.38 (m, 4H), 2.65-2.86 ppm (m, 6H); MS m/e 510 (M+H).

### Example 9

### 2-Amino-4-(4-fluoro-phenyl)-9-[4-(4-isopropyl-piperazin-1-yl)-phenyl]-indeno[1,2-d]pyrimidin-5-one

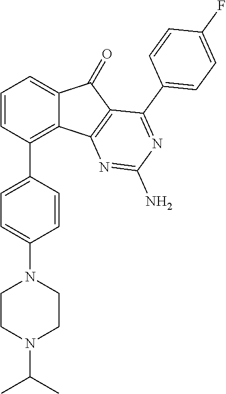

The title compound was prepared using 2-iodo-propane in place of 1,1,1-trifluoro-3-iodo-propane as described in Example 1. ¹H NMR(CHLOROFORM-d, 300 MHz): δ=8.03-8.14 (m, 2H), 7.69 (dd, J=6.4, 1.9 Hz, 1H), 7.47-7.59 (m, 4H), 7.11-7.22 (m, 2H), 7.01 (d, J=8.7 Hz, 2H), 5.43 (br. s., 2H), 3.28-3.39 (m, 4H), 2.74 (br. s., 4H), 1.52-1.65 (m, 1H), 1.13 ppm (d, J=6.4 Hz, 6H); MS m/e 494 (M+H).

### Example 10

### 4-[2-Amino-4-(4-fluoro-phenyl)-5-oxo-5H-indeno[1,2-d]pyrimidin-9-yl]-benzonitrile

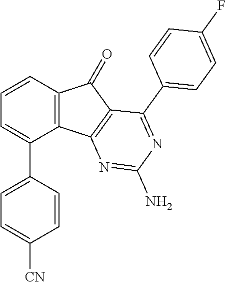

The title compound was prepared using 4-cyano-phenylboronic acid in place of 3-fluoro-phenylboronic acid as described in Example 3. ¹H NMR (DMSO-d₆, 400 MHz): δ=8.06-8.14 (m, 2H), 7.96 (d, J=8.3 Hz, 2H), 7.88 (d, J=8.3 Hz, 2H), 7.76-7.82 (m, 2H), 7.64-7.71 (m, 1H), 7.40 ppm (t, J=8.9 Hz, 2H); MS m/e 393 (M+H).

### Example 11

### 2-Amino-4-(4-fluoro-phenyl)-9-{4-[4-(3-methyl-butyl)-piperazin-1-yl]-phenyl}-indeno[1,2-d]pyrimidin-5-one

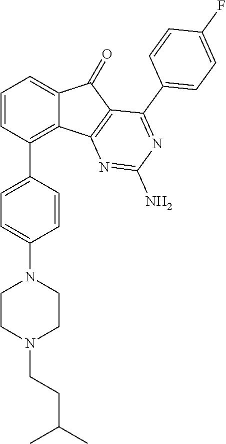

The title compound was prepared using 1-iodo-3-methyl-butane in place of 1,1,1-trifluoro-3-iodo-propane as described in Example 1. ¹H NMR(CHLOROFORM-d, 300 MHz): δ=8.01-8.14 (m, 2H), 7.69 (dd, J=6.6, 1.7 Hz, 1H), 7.44-7.59 (m, 4H), 7.12-7.22 (m, 2H), 7.01 (d, J=9.0 Hz, 2H), 5.46 (br. s., 2H), 3.29-3.36 (m, 4H), 2.61-2.70 (m, 4H), 2.40-2.48 (m, 2H), 1.41-1.51 (m, 2H), 1.21-1.30 (m, 1 H), 0.94 ppm (d, J=6.8 Hz, 6H); MS m/e 522 (M+H).

### Example 12

### 2-Amino-9-[4-(4-ethyl-piperazin-1-yl)-phenyl]-4-(4-fluoro-phenyl)-indeno[1,2-d]pyrimidin-5-one

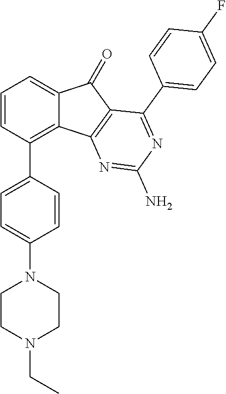

The title compound was prepared using iodo-ethane in place of 1,1,1-trifluoro-3-iodo-propane as described in Example 1. ¹H NMR(CHLOROFORM-d, 300 MHz): δ=8.03-8.14 (m, 2H), 7.69 (dd, J=6.6, 1.7 Hz, 1H), 7.52 (dt, J=8.9, 6.1 Hz, 4H), 7.12-7.21 (m, 2H), 7.01 (d, J=8.7 Hz, 2H), 5.45 (br. s., 2H), 3.27-3.40 (m, 4H), 2.61-2.73 (m, 4H), 2.44-2.58 (m, 2H), 1.16 ppm (t, J=7.3 Hz, 3H); MS m/e 480 (M+H).

### Example 13

### 2-Amino-4-(4-fluoro-phenyl)-9-[4-(2-morpholin-4-yl-ethoxy)-phenyl]-indeno[1,2-d]pyrimidin-5-one

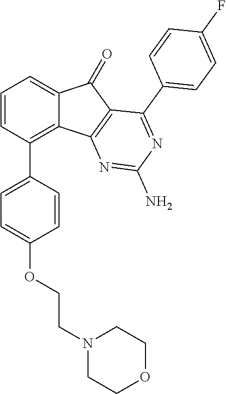

The title compound was prepared using 4-(2-morpholinoethoxy)phenylboronic acid in place of 1-[4-(4,4,5,5-tetramethyl-[1,3,2]dioxaborolan-2-yl)-phenyl]-piperazine as described in Example 1. ¹H NMR(CHLOROFORM-d, 300 MHz): δ=8.00-8.18 (m, 2H), 7.72 (d, J=7.2 Hz, 1H), 7.42-7.61 (m, 4H), 7.10-7.23 (m, 2H), 6.93-7.06 (m, 2H), 5.43 (br. s., 2H), 4.21 (t, J=5.7 Hz, 2H), 3.73-3.82 (m, 4H), 2.87 (t, J=5.7 Hz, 2H), 2.57-2.68 ppm (m, 4H); MS m/e 497 (M+H).

### Example 14

### 2-Amino-4,9-diphenyl-indeno[1,2-d]pyrimidin-5-one

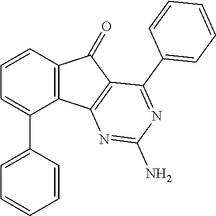

The title compound was prepared using phenylboronic acid in place of 3-fluoro-phenylboronic acid as described in Example 3. ¹H NMR (DMSO-d₆, 400 MHz): δ=7.88-7.96 (m, 2H), 7.41-7.73 ppm (m, 12H); MS m/e 350 (M+H).

### Example 15

### 2-Amino-4-(4-fluoro-phenyl)-9-[4-(4-propyl-piperazin-1-yl)-phenyl]-indeno[1,2-d]pyrimidin-5-one

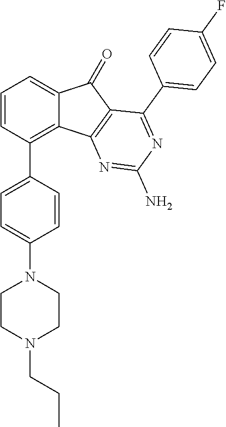

The title compound was prepared using 1-iodo-propane in place of 1,1,1-trifluoro-3-iodo-propane as described in Example 1. ¹H NMR(CHLOROFORM-d, 300 MHz): δ=8.04-8.13 (m, 2H), 7.69 (dd, J=6.8, 1.9 Hz, 1H), 7.47-7.58 (m, 4H), 7.12-7.22 (m, 2H), 6.96-7.04 (m, 2H), 5.44 (br. s., 2H), 3.28-3.38 (m, 4H), 2.60-2.70 (m, 4H), 2.34-2.44 (m, 2H), 1.51-1.64 (m, 2H), 0.95 ppm (t, J=7.3 Hz, 3H); MS m/e 494 (M+H).

### Example 16

### 2-Amino-4-(4-fluoro-phenyl)-9-m-tolyl-indeno[1,2-d]pyrimidin-5-one

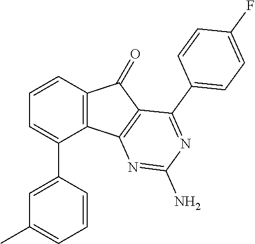

The title compound was prepared using 3-methyl-phenylboronic acid in place of 3-fluoro-phenylboronic acid as described in Example 3. ¹H NMR (DMSO-d₆, 400 MHz): δ=7.99-8.08 (m, 2H), 7.63-7.71 (m, 2H), 7.52-7.60 (m, 1H), 7.38-7.46 (m, 2 H), 7.29-7.38 (m, 3H), 7.24 (d, J=7.6 Hz, 1H), 2.40 ppm (s, 3H); MS m/e 382 (M+H).

### Example 17

### 2-Amino-9-(4-methoxy-phenyl)-4-phenyl-indeno[1,2-d]pyrimidin-5-one

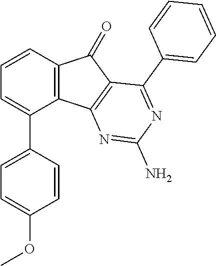

The title compound was prepared using 4-methoxy-phenylboronic acid in place of 3-fluoro-phenylboronic acid as described in Example 3. ¹H NMR (DMSO-d₆, 400 MHz): δ=7.93 (dd, J=8.3, 1.5 Hz, 2H), 7.46-7.69 (m, 9H), 6.99-7.05 (m, 2H), 3.84 ppm (s, 3H); MS m/e 380 (M+H).

### Biological Assays and Activity

### Lip and Binding Assay for Adenosine A_{2A} Receptor

Ligand binding assay of adenosine A_{2A} receptor was performed using plasma membrane of HEK293 cells containing human A_{2A} adenosine receptor (PerkinElmer, RB-HA_{2A}) and radioligand [³H]CGS21680 (PerkinElmer, NET1021). Assay was set up in 96-well polypropylene plate in total volume of 200 μl by sequentially adding 20 μL 1:20 diluted membrane, 130 μL assay buffer (50 mM Tris.HCl, pH7.4 10 mM MgCl₂, 1 mM EDTA) containing [³H] CGS21680, 50 μL diluted compound (4×) or vehicle control in assay buffer. Nonspecific binding was determined by 80 mM NECA. Reaction was carried out at room temperature for 2 hours before filtering through 96-well GF/C filter plate pre-soaked in 50 mM Tris.HCl, pH7.4 containing 0.3% polyethylenimine. Plates were then washed 5 times with cold 50 mM Tris.HCl, pH7.4, dried and sealed at the bottom. Microscintillation fluid 30 μL was added to each well and the top sealed. Plates were counted on Packard Topcount for [³H]. Data was analyzed in Microsoft Excel and GraphPad Prism programs. (Varani, K.; Gessi, S.; Dalpiaz, A.; Borea, P. A. British Journal of Pharmacology, 1996, 117, 1693)

### Adenosine A_{2A} Receptor Functional Assay (A_{2A}GAL2)

To initiate the functional assay, cryopreserved CHO-K1 cells overexpressing the human adenosine A_{2A} receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 μL assay medium (Hams/F-12 Modified (Mediatech # 10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 15 nM NECA (Sigma E2387) agonist challenge (5 μL volume). A control curve of NECA, a DMSO/Media control, and a single dose of Forskolin (Sigma F3917) were also included on each plate. After additions, cell plates were allowed to incubate at 37° C., 5% CO₂, 90% Rh for 5.5-6 hours. After incubation, media were removed, and cell plates were washed 1×50 μL with DPBS w/o Ca & Mg (Mediatech 21-031-CV). Into dry wells, 20 μL of 1× Reporter Lysis Buffer (Promega E3971 (diluted in dH₂O from 5× stock)) was added to each well and plates frozen at −20° C. overnight. For β-galactosidase enzyme colorimetric assay, plates were thawed out at room temperature and 20 μL 2× assay buffer (Promega) was added to each well. Color was allowed to develop at 37° C., 5% CO₂, 90% Rh for 1-1.5 hours or until reasonable signal appeared. The colorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

### Adenosine A1 Receptor Functional Assay (A1GAL2)

To initiate the functional assay, cryopreserved CHO-K1 cells overexpressing the human adenosine A1 receptor and containing a cAMP inducible beta-galactosidase reporter gene were thawed, centrifuged, DMSO containing media removed, and then seeded with fresh culture media into clear 384-well tissue culture treated plates (BD #353961) at a concentration of 10K cells/well. Prior to assay, these plates were cultured for two days at 37° C., 5% CO₂, 90% Rh. On the day of the functional assay, culture media was removed and replaced with 45 μL assay medium (Hams/F-12 Modified (Mediatech # 10-080CV) supplemented w/0.1% BSA). Test compounds were diluted and 11 point curves created at a 1000× concentration in 100% DMSO. Immediately after addition of assay media to the cell plates, 50 nL of the appropriate test compound antagonist or agonist control curves were added to cell plates using a Cartesian Hummingbird. Compound curves were allowed to incubate at room temperature on cell plates for approximately 15 minutes before addition of a 4 nM r-PIA (Sigma P4532)/1 uM Forskolin (Sigma F3917) agonist challenge (5 μL volume). A control curve of r-PIA in 1 uM Forskolin, a DMSO/Media control, and a single dose of Forskolin were also included on each plate. After additions, cell plates were allowed to incubate at 37° C., 5% CO₂, 90% Rh for 5.5-6 hours. After incubation, media was removed, and cell plates were washed 1×50 μL with DPBS w/o Ca & Mg (Mediatech 21-031-CV). Into dry wells, 20 μL of 1× Reporter Lysis Buffer (Promega E3971 (diluted in dH₂O from 5× stock)) was added to each well and plates frozen at −20° C. overnight. For β-galactosidase enzyme colorimetric assay, plates were thawed out at room temperature and 20 μL 2× assay buffer (Promega) was added to each well. Color was allowed to develop at 37° C., 5% CO₂, 90% Rh for 1-1.5 hours or until reasonable signal appeared. The colorimetric reaction was stopped with the addition of 60 μL/well 1M sodium carbonate. Plates were counted at 405 nm on a SpectraMax Microplate Reader (Molecular Devices). Data was analyzed in Microsoft Excel and IC/EC50 curves were fit using a standardized macro.

### A_{2A} Assay Data

Compounds of Formula A displayed surprising and unexpected selectivity for A_{2A} over A1 receptor antagonism.

| Example | A_{2A}Gal2 (μM) | A1Gal2 (μM) | A1/A_{2A} |
|---|---|---|---|
| 1 | 0.0018 | 5.5 | 3055.56 |
| 2 | 0.0071 | 4.2 | 591.549 |
| 3 | 0.026 | 8.9 | 342.308 |
| 4 | 0.01 | 3.1 | 310 |
| 5 | 0.023 | 4.2 | 182.609 |
| 6 | 0.0098 | 1.4 | 142.857 |
| 7 | 0.0083 | 1.1 | 131.69 |
| 8 | 0.0018 | 0.23 | 127.778 |
| 9 | 0.016 | 1.8 | 112.5 |
| 10 | 0.0026 | 0.27 | 103.846 |
| 11 | 0.011 | 1.1 | 100 |
| 12 | 0.01 | 0.76 | 76 |
| 13 | 0.0039 | 0.27 | 69.2308 |
| 14 | 0.0086 | 0.57 | 66.2791 |
| 15 | 0.0087 | 0.52 | 59.7701 |
| 16 | 0.031 | 1.8 | 58.0645 |
| 17 | 0.0063 | 0.33 | 52.381 |

While the foregoing specification teaches the principles of the present invention, with examples provided for the purpose of illustration, it will be understood that the practice of the invention encompasses all of the usual variations, adaptations and/or modifications as come within the scope of the following Claims and their equivalents.

All publications disclosed in the above specification are hereby incorporated by reference in full.

We claim:

1. Arylindenopyrimidines of Formula A: 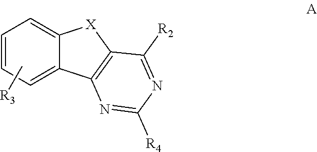
wherein:
X is C═O;
R₂ is phenyl;
R₄ is NH₂; and
R₃ is aryl;
said arylindenopyrimidines of Formula A are selected form the group consisting of: 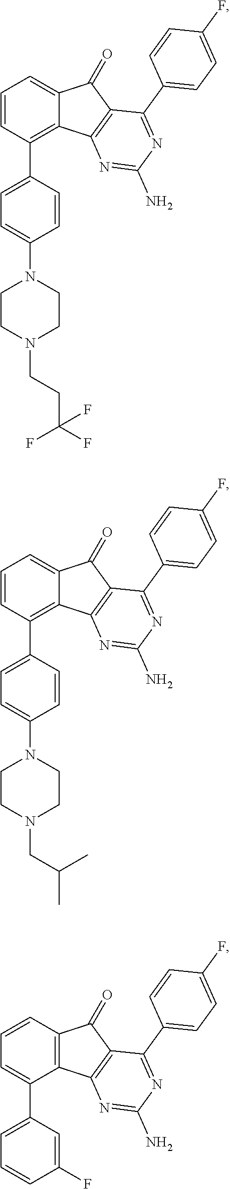 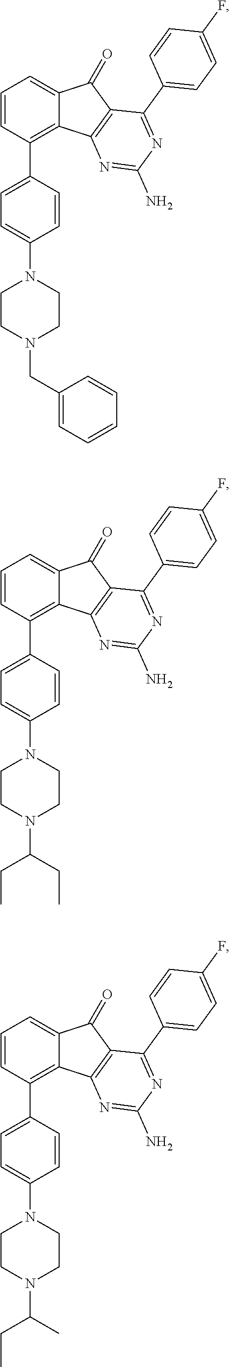 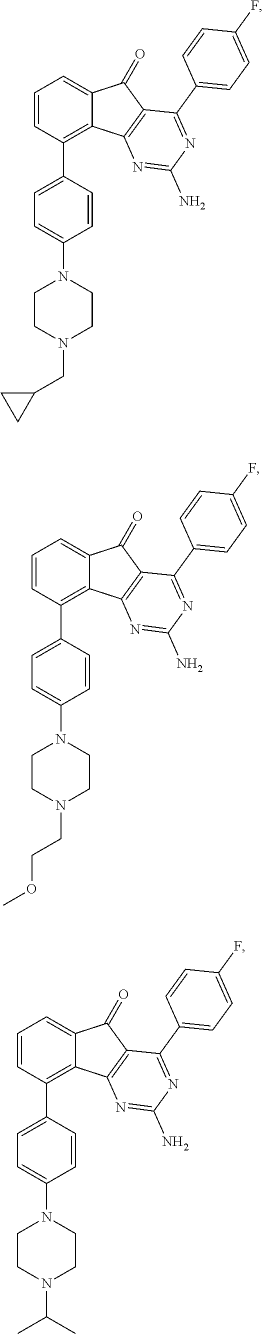 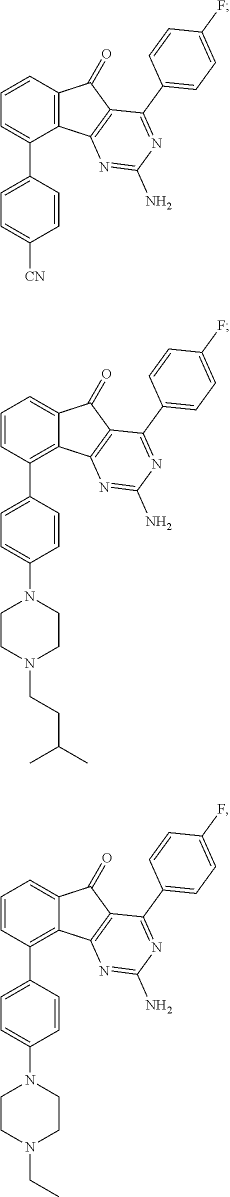 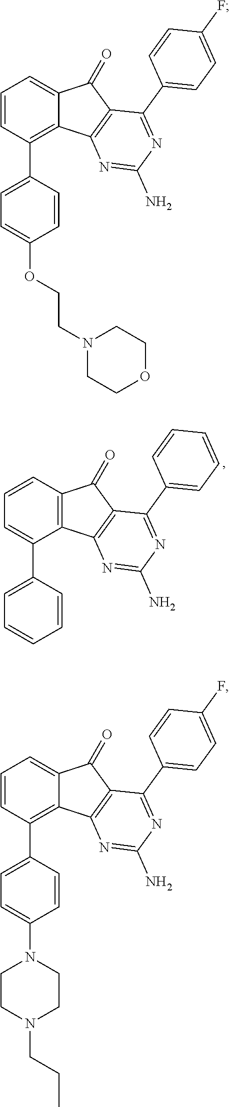 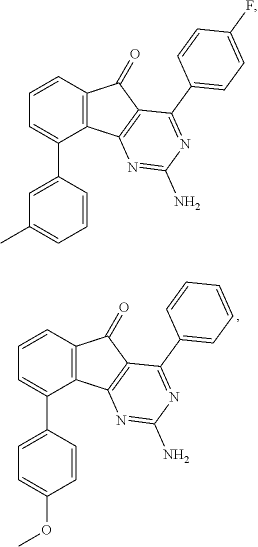
and solvates, hydrates, tautomers, and pharmaceutically acceptable salts thereof;

2. A pharmaceutical composition comprising a compound of claim 1 and a pharmaceutically acceptable carrier.

3. A method of treating a subject having a disorder ameliorated by antagonizing Adenosine A_{2A} receptors in appropriate cells in the subject, which comprises administering to the subject a therapeutically effective dose of a compound of claim 1.

4. A method of preventing a disorder ameliorated by antagonizing Adenosine A_{2A} receptors in appropriate cells in the subject, comprising administering to the subject a prophylactically effective dose of a compound of claim 1 either preceding or subsequent to an event anticipated to cause a disorder ameliorated by antagonizing Adenosine A_{2A} receptors in appropriate cells in the subject.

5. The method of claim 3 comprising administering to the subject a therapeutically or prophylactically effective dose of the pharmaceutical composition of claim 2.

6. The method of claim 4 comprising administering to the subject a therapeutically or prophylactically effective dose of the pharmaceutical composition of claim 2.

7. The method of claim 3, wherein the disorder is a neurodegenerative disorder or a movement disorder.

8. The method of claim 3, wherein the disorder is selected from the group consisting of Parkinson's Disease, Huntington's Disease, Multiple System Atrophy, Corticobasal Degeneration, Alzheimer's Disease, or Senile Dementia.

9. The method of claim 4, wherein the disorder is a neurodegenerative disorder or a movement disorder.

10. The method of claim 4, wherein the disorder is selected from the group consisting of Parkinson's Disease, Huntington's Disease, Multiple System Atrophy, Corticobasal Degeneration, Alzheimer's Disease, or Senile Dementia.

11. The method of claim 3, wherein the disorder is Parkinson's Disease.

12. The method of claim 3, wherein the disorder is addiction.

13. The method of claim 3, wherein the disorder is Attention Deficit Hyperactivity Disorder (ADHD).

14. The method of claim 3, wherein the disorder is depression.

15. The method of claim 3, wherein the disorder is anxiety.

16. The method of claim 3, wherein the disorder is migraine.
